(19) United States
(12) Patent Application Publication
(10) Pub. No.: US 2007/0010423 A1
(43) Pub. Date: Jan. 11, 2007

(54) Compositions comprising balaglitazone and further antidiabetic compounds

(76) Inventors: Karsten Wassermann, Belle Mead, NJ (US); Erik Max Wulff, Birkerod (DK)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 10/561,639

(22) PCT Filed: Jun. 24, 2004
(86) PCT No.: PCT/DK04/00448

§ 371 Date: Sep. 22, 2006

Related U.S. Application Data
(60) Provisional application No. 60/483,196, filed on Jun. 27, 2003.

(30) Foreign Application Priority Data
Jun. 27, 2003 (DK) PA 2003 00973

(51) Int. Cl. A61K 38/28; A61K 31/4439; A61K 31/426; A61K 31/401; A61K 31/366; A61K 31/22; A61K 31/155

(52) U.S. Cl. 514/3; 514/342; 514/369; 514/594; 514/423; 514/460; 514/548; 514/635

(57) ABSTRACT

Methods for the treatment of type 2 diabetes and related conditions comprising the admini-stration of balaglitazone in combination with one or more other antidiabetic compound is pro-vided together with combinations useful in said treatment.

### FIELD OF THE INVENTION

The invention relates to novel combinations of balaglitazone and other anti-diabetic compounds, said combination being particular well-suited for the treatment of type 2 diabetes and related conditions.

### BACKGROUND OF THE INVENTION

Diabetes, and in particular type 2 diabetes is a disease with a large an ever increasing number of incidences, and treatment of diabetes, and in particular type 2 diabetes and related conditions is a main challenge to health authorities in major parts of the world.

Type 2 diabetes is characterised by peripheral insulin resistance, increased insulin secretion and increased hepatic glucose output. Related conditions often encountered in combination with type 2 diabetes, either as part of the etiology or as a complication includes dyslipidemia, hyperglycemia, hyperinsulinemia, impaired glucose tolerance, impaired fasting glucose, increased plasma levels of free fatty acids, increased plasma levels of triglycerides, e.g. in the form of very low density lipoproteins (VLDL), obesity, hypertension and cardiovascular complications, such as atherosclerosis.

Thiazolidinediones (TZD) is a group of compounds which have proved to be useful in the treatment of diabetes and related conditions. TZD exert their function by activating the peroxisome proliferators activated receptor γ (PPARγ), which is a receptor present in tissue with metabolic significance, e.g. adipose tissue, skeletal muscles and in the liver. An activation of PPARγ results in a reduction of insulin resistance and a lowering of the plasma glucose level, and PPARγ activators (also termed PPARγ agonists) thus belongs to the group of insulin sensitizers.

Balaglitazone, which is the potassium salt of 5[4-(3-methyl-4-oxo-3,4-dihydro-quinazolin-2-ylmethoxy)-benzyl]-thiazolidine-2,4-dione, is a new PPARγ agonist.

It is an aim of the present invention to provide combinations comprising balaglitazone together with one or more other anti-diabetic compounds, as well as methods for treatment of type 2 diabetes or related conditions comprising the administration of said combinations.

Balaglitazone was first disclosed in WO 97/41097, and combinations of insulin sensitizers in general and TZD in particular and other anti-diabtec compounds have also been disclosed. U.S. Pat. No. 6,153,632 discloses compositions comprising an insulin sensitizer and an anti-diabetic agent. U.S. Pat. No. 6,150,383, U.S. Pat. No. 6,211,205 and U.S. Pat. No. 6,303,640 all disclose different therapeutic methods comprising the administration of TZD and insulin secretagogues. WO 98/36755 discloses a synergistic combination of TZD and sulfonylureas. WO 02/72146 discloses combinations of nateglinide or repaglinide with e.g. PPARγ/PPARα agonists. WO 00/45818 discloses therapeutic interventions comprising the administration of statin in combination with other anti-diabetic drugs. WO 01/32158 discloses the combination of metformin and an insulin sensitizer. WO 00/78333 discloses the combination of TZD and incretin hormones, e.g. GLP-1. WO 97/10819 discloses the use TZD in combination with RXR agonists to control the level of HDL cholesterol. WO 00/38666 discloses the use of combination of fructose-1,6-biphophatase inhibitor and insulin sensitizer to treat diabetes. WO 01/52825 discloses the combinations of dipeptyl peptidase inhibitors and other anti-diabetic agents. WO 01/03659 discloses the combination of RAR antagonists and PPAR agonists. WO 02/058732 discloses the combination of PPAR activators in combination with sterol absorption inhibitors useful in the treatment of vascular indications. WO 02/13864 discloses the combination of a PPARγ agonist and an RXR agonist useful in the treatment of cancer. WO 98/57635 discloses the use if a combination of an insulin sensitizer and an α-glucosidase inhibitor. WO 00/27434 discloses the use of a combination of insulin sensitizers and β-adrenorecptor agonists. WO 97/37688 discloses a combination of insulin sensitizers and angiotensin antagonists useful in the treatment of hyperglycemia. WO 00/00195 discloses the use of compositions of insulin sensitizers and anorectic compounds. U.S. Pat. No. 6,133,293, U.S. Pat. No. 6,211,206 and U.S. Pat. No. 6,211,207 disclose the use of combinations of insulin sensitizers with fibrates to treat various diabetic complications. U.S. Pat. No. 6,169,099, U.S. Pat. No. 6,251,924, U.S. Pat. No. 6,239,153 and U.S. Pat. No. 6,323,225 disclose the combination of insulin sensitizers and squalene synthesis inhibitors for various aspects of diabetes care. U.S. Pat. No. 6,214,848 discloses the combination of an LDL catabolism enhancer with insulin sensitizers useful in the treatment of diabetes. U.S. Pat. No. 6,172,089, U.S. Pat. No. 6,274,605 and U.S. Pat. No. 6,277,869 disclose the combination of ACE inhibitors and insulin sensitizers useful in the treatment of diabetic complications. U.S. Pat. No. 6,156,773, U.S. Pat. No. 6,218,409, U.S. Pat. No. 6,232,330 and U.S. Pat. No. 6,288,090 disclose combinations of aldose reductase inhibitors and insulin sensitizers useful in the treatment of diabetes. WO 02/72069 discloses a particular formulation comprising balaglitazone and optionally an anti-diabetic agent.

### SUMMARY OF THE INVENTION

The present inventors have surprisingly found that combinations of balaglitazone and one or more other therapeutically active compounds, and in particular other anti-diabetic compounds show an unexpected advantage over prior art. The present invention thus provides the combination of balaglitazone and one or more other therapeutically active compounds, and in particular other anti-diabetic compounds.

In a further aspect, the present invention provides a method of treating type 2 diabetes or related conditions, the method comprising administering a combination of balaglitazone and one or more another therapeutically active compounds, and in particular other antidiabetic compounds to a patient in need thereof.

In a still further aspect, the invention relates to the use of balaglitazone and one or more another therapeutically active compound, and in particular another anti-diabetic compound in the manufacture of a medicament for the treatment of type 2 diabetes or related conditions.

Definitions

In the present context the term “obese” or “obesity” implies an excess of adipose tissue. The term can only be approximated, but in the present context, any person with a bodymass index (BMI=body weight in kg divided by the square of the height in meters) above 25 is regarded as obese.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, paminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs.

In the present context, the term “prodrug” is intended to indicate a compound which does not, or which does not necessarily have a therapeutic activity, but which upon administration is transformed in the body to the therapeutically active compound. Often this transformation relies on enzymatic activities in the body or on acid-base catalysed reactions in the intestines.

### DESCRIPTION OF THE INVENTION

In one aspect, the present invention relates to a method of treating type 2 diabetes or related conditions, the method comprising administering to a patient in need thereof an effective amount of balaglitazone in combination with one or more other therapeutically active compounds, and in particular another anti-diabetic compounds.

In one aspect, said conditions are selected from amongst dyslipidemia, hyperglycemia, hyperinsulinemia, insulin resistance, obesity, cardiovascular complications, such as atherosclerosis, hypertension, impaired glucose tolerance, impaired fasting glucose level, increased plasma levels of free fatty acids, increased plasma level of triglycerides, increased plasma levels of very low density lipoproteins (VLDL).

In one aspect, the invention relates to a method of increasing the plasma level of high density lipoproteins at the expense of the plasma level of VLDL, of decreasing the plasma glucose level, of decreasing the plasma level of free fatty acids, of decreasing the plasma level of triglyceride, of delaying the progression of impaired glucose tolerance to non-insulin requiring type 2 diabetes, or of delaying the progression of non-insulin requiring type 2 diabetes to insulin requiring type 2 diabetes, the method comprising administering to a patient in need thereof an effective amount of balaglitazone in combination with one or more other therapeutically active compounds, and in particular another anti-diabetic compounds.

In another aspect, the invention relates to a method of treating a disease benefiting from a lowering of the plasma glucose level, a lowering of the plasma free fatty acid level, a lowering of the plasma level of triglyceride or a lowering of the plasma VLDL level, the method comprising administering to a patient in need thereof as effective amount of balaglitazone in combination with one or more other anti-diabetic compound.

In one aspect of all the methods of the present invention, the patient is obese.

In one aspect, the other therapeutically active compound, and in particular the other anti-diabetic compound is selected from amongst insulin together with derivative and analogues thereof, insulin secretagogues (also called insulin secretion enhancers and insulinotropic agents), insulin sensitizers, biguanides, α-glucosidase inhibitors, potassium channel openers, glucagon antagonists, protein tyrosine phosphatase inhibitors, glucokinase activators, RXR agonists, hormone sensitive lipase inhibitors, glycogen synthase kinase-3 inhibitors, glycogen phosphorylase inhibitors, glucose uptake modulators and lipid lowering compounds.

Useful insulin and derivatives and analogues thereof include human insulin and and derivatives and analogues thereof. The term human insulin as used herein refers to naturally produced insulin or recombinantly produced insulin. Recombinant human insulin may be produced in any suitable host cell, for example the host cells may be bacterial, fungal (including yeast), insect, animal or plant cells. The expression “insulin derivative” as used herein refers to human insulin or an analogue thereof in which at least one organic substituent is bound to one or more of the amino acids. By “analogue of human insulin” as used herein (and related expressions) is meant human insulin in which one or more amino acids have been deleted and/or replaced by other amino acids, including non-codeable amino acids, or human insulin comprising additional amino acids, i.e. more than 51 amino acids, such that the resulting analogue possesses insulin activity.

Particular useful insulin analogues are those disclosed in EP 792 290, EP 214 826, EP 705 275 (Novo Nordisk A/S) and U.S. Pat. No. 5,504,188, e.g. B28 Lys-B29 Pro human insulin and EP 368 187, e.g. Lantus®, all of which are incorporated herein be reference.

Particular useful insulin analogues include an analogue wherein position B28 is Asp, Lys, Leu, Val, or Ala and position B29 is Lys or Pro; des(B28-B30), des(B27) and des(B30) human insulin. Useful insulin derivatives include human insulin derivatives selected from amongst B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28}Pro^{B29} human insulin, B28-N^{ε}-palmitoyl Lys^{B28}Pro^{B29} human insulin, B30-N^{ε}-myristoyl-Thr^{B29}Lys^{B30} human insulin, B30-N-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-((ω-carboxyheptadecanoyl)-des(B30) human insulin and B29-N^{ε}-((ω-carboxyheptadecanoyl) human insulin.

Useful insulin secretagogues include sulfonylureas, meglitinides and dipeptidyl peptidase inhibitors.

Useful sulfonylureas include tolbutamide, glibenclamide, gliclazide, glimepiride, glipizid, chlorpropamide, tolazamide and glyburide.

Useful meglitinides include nateglinide and repaglinide.

Useful dipeptidyl peptidase inhibitors include compounds disclosed in D 296075 (Martin-Luther-Universität), WO 91/16339 and WO 93/08259 (New England Medical Centre Hospitals, Inc. and Tufts University School of Medicine), WO 95/15309, WO 01/40180, WO 01/81337 and WO 01/81304 (Ferring B.V.), WO 98/19998, U.S. Pat. No. 6,110,949, WO 00/34241 and WO 01/96295 (Novartis AG), WO 99/46272 (Fondatech Benelux N.V.), WO 99/61431, WO 99/67278, WO 99/67279 and WO 01/14318 (Probiodrug Gesellschaft für Artzneimittelforschung Mbh.), WO 01/55105, WO 02/02560, WO 03/024965 and WO 03/04496 (Novo Nordisk A/S) or WO 01/68603 (Bristol-Myers Squibb Co.), all of which are incorporated herein by reference.

Useful insulin sensitizers include TZD insulin sensitizer e.g. troglitazone, ciglitazone, pioglitazone, rosiglitazone, isaglitazone, darglitazone, englitazone, CS-011/CI-1037 or T 174 or the compounds disclosed in WO 97/41097 (excluding balaglitazone), WO 97/41119, WO 97/41120, WO 00/41121 and WO 98/45292, all of which are incorporated herein by reference. Non-TZD insulin sensitizers include GI 262570, YM-440, MCC-555, JTT-501, AR-H039242, KRP-297, GW409544, CRE-16336, AR-H049020, LY510929, MBX-102, CLX-0940, GW-501516 or the compounds disclosed in WO 99/19313, WO 00/50414, WO 00/63191, WO 00/63192, WO 00/63193, WO 00/23425, WO 00/23415, WO 00/23451, WO 00/23445, WO 00/23417, WO 00/23416, WO 00/63153, WO 00/63196, WO 00/63209, WO 00/63190 and WO 00/63189, all of which are incorporated herein by reference.

Useful biguanides include metformin.

Useful α-glucosidase inhibitors include voglobose, emiglitate, miglitol and acarbose.

Useful potassium channel openers include diazoxide and compounds disclosed in WO 97/26265, WO 99/03861 and WO 00/37474, all of which are incorporated herein by reference.

Useful glucagon antagonist include compounds disclosed in WO 99/01423 and WO 00/39088, both of which are incorporated herein by reference Useful glucokinase activators include compounds disclosed in WO 02/08209, WO 00/58293, WO 01/44216, WO 01/83465, WO 01/83478, WO 01/85706, WO 01/85707, and WO 02/08209, WO03/00262, WO 03/00267 and WO 03/15774, all of which are incorporated herein by reference.

Useful RXR (retinoid X receptor) agonists include ALRT-268, LG-1268 or LG-1069.

Useful hormone sensitive lipase inhibitors include compounds disclosed in PCT/DK02/00852, which is incorporated herein by reference.

Useful glycogen phosphorylase inhibitors include compounds disclosed in WO 97/09040, which is incorporate herein by reference.

Useful lipid lowering compounds include statins, fibrates and PPARδ agonists.

Useful statins include atorvastatin, lovastatin, pravastatin, simvastatin, fluvastatin and cerivastatin.

Useful fibrates include fenofibrate, gemfibrozil, bezafibrate and any other PPARα agonist.

As appropriate, the other anti-diabetic compounds may be used in the form of the free acid or base rather than as a salt, or as a pharmaceutically acceptable salt rather than as a free acid or base. The use of prodrugs or solvates of said other anti-diabetic compounds is also part of the present invention

Very often a treatment of type 2 diabetes and related conditions includes diet and exercise. The present invention also includes any of the above mentioned methods of treatment in any combination with diet and/or exercise.

It is a common feature of the methods of the present invention that balaglitazone and the other anti-diabetic compounds may be administered either concomitantly or sequentially.

In another aspect, the invention provides combinations of balaglitazone and any of the above mentioned anti-diabetic compounds useful in the above mentioned therapeutic methods.

One embodiment of the invention provides a pharmaceutical composition comprising balaglitazone and any of the above mentioned anti-diabetic compounds.

One embodiment of the invention provides balaglitazone and any of the above mentioned anti-diabetic compounds presented in two or more separate containers intended for sequentially or concomitantly administration.

In another aspect, the invention relates to the use of balaglitazone and any of the above mentioned anti-diabetic compounds in the manufacture of a medicament for the treatment of type 2 diabetes, dyslipidemia, hyperglycemia, hyperinsulinemia, insulin resistance, obesity, cardiovascular complications, such as atherosclerosis, hypertension, impaired glucose tolerance, impaired fasting glucose level, increased plasma levels of free fatty acids, increased plasma level of triglycerides, increased plasma levels of very low density lipoproteins (VLDL) or increased plasma triglyceride levels, or for increasing the plasma level of high density lipoproteins at the expense of the plasma level of VLDL, decreasing the plasma glucose level, decreasing the plasma level of free fatty acids, decreasing the plasma triglyceride level, delaying the progression of impaired glucose tolerance to non-insulin requiring type 2 diabetes or delaying the progression of non-insulin requiring type 2 diabetes to insulin requiring type 2 diabetes.

In another aspect, the invention relates to the use of balaglitazone and any of the above mentioned anti-diabetic compounds in the manufacture of a medicament for the treatment of a disease benefiting from a lowering of the plasma glucose level, a lowering of the plasma free fatty acid level, a lowering of the plasma triglyceride level or a lowering of the plasma VLDL level.

In a further aspect, this invention also relates to the use of 5[4-(3-methyl-4-oxo-3,4-dihydro-quinazolin-2-ylmethoxy)-benzyl]-thiazolidine-2,4-dione itself, or any pharmaceutically acceptable salt, prodrug or solvate thereof.

Pharmaceutical Compositions

The compounds for use according to the present invention may be administered alone or in combination with pharmaceutically acceptable carriers or excipients, in either single or multiple doses. The pharmaceutical compositions according to the present invention may be formulated with pharmaceutically acceptable carriers or diluents as well as any other known adjuvants and excipients in accordance with conventional techniques such as those disclosed in Remington: The Science and Practice of Pharmacy, 20^{th} Edition, Gennaro, Ed., Mack Publishing Co., Easton, Pa., 2000.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

The compounds for use according to the present invention are generally utilized as the free substance or as a pharmaceutically acceptable salt thereof. Examples are an acid addition salt of a compound having the utility of a free base and a base addition salt of a compound having the utility of a free acid.

For parenteral administration, solutions of the compounds for use according to the present invention in sterile aqueous solution, aqueous propylene glycol or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds for use according to the present invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsule wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising a compound for use according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the present invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the present invention are contemplated. For the purpose of this application, topical applications shall include mouth washes and gargles.

The compounds for use according to the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some of the compounds for use according to the present invention may form solvates with water or common organic solvents. Such solvates are also encompassed within the scope of the present invention.

Thus, in a further embodiment, there is provided a pharmaceutical composition comprising a compound for use according to the present invention, or a pharmaceutically acceptable salt, solvate, or prodrug thereof, and one or more pharmaceutically acceptable carriers, excipients, or diluents.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

1. A pharmaceutical composition comprising balaglitazone and one or more other anti-diabetic compounds.

2. A composition according to claim 1, wherein said anti-diabetic compound is selected from amongst insulin together with derivative and analogues thereof, insulin secretagogues, insulin sensitizers, biguanides α-glucosidase inhibitors, potassium channel openers, glucagons antagonists, protein tyrosine phosphatase inhibitors, glucokinase activators, RXR agonists, hormone sensitive lipase inhibitors, glycogen synthase kinase-3 inhibitors, glycogen phosphorylase inhibitors, glucose uptake modulators and lipid lowering compounds.

3. A composition according to claim 2, wherein said insulin, insulin analogue or insulin derivative is selected from amongst human insulin, human insulin wherein position B28 is Asp, Lys, Leu, Val or Ala and position B29 is Lys or Pro, B28Lys-B29Pro human insulin, des(B28-B30) human insulin, des(B27) human insulin, des(B30) human insulin, B29-N^{ε}-myristoyl-des(B30) human insulin, B29-N-palmitoyl-des(B30) human insulin, B29-N^{ε}-myristoyl human insulin, B29-N-palmitoyl human insulin, B28-N^{ε}-myristoyl Lys^{B28}Pro^{B29} human insulin, B28-N^{ε}-palmitoyl Lys^{B28}Pro^{B29} human insulin, B30-N^{ε}-myristoyl-Thr^{B29}Lys^{B30} human insulin, B30-N^{ε}-palmitoyl-Thr^{E29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

4. A composition according to claim 2, wherein said insulin secretagogue is selected from amongst sulfonylureas, meglitinides and dipeptidyl peptidase inhibitors.

5. A composition according to claim 4, wherein said sulfonylurea is selected from amongst tolbutamide, glibenclamide, gliclazide, glimepiride, glipizid, chlorpropamide, tolazamide and glyburide.

6. A composition according to claim 4, wherein said meglitinides is selected from amongst nateglinide and repaglinide.

7. A composition according to claim 2, wherein said insulin sensitizer is selected from amongst troglitazone, ciglitazone, pioglitazone, rosiglitazone, isaglitazone, darglitazone, englitazone.

8. A composition according to claim 2, wherein said biguanide is metformin.

9. A composition according to claim 2, wherein said α-glucosidase inhibitor is selected from amongst voglobose, emiglitate, miglitol and acarbose.

10. A composition according to claim 2, wherein said lipid lowering compound is a statin, fibrate or a PPARδ agonist.

11. A composition according to claim 10, wherein said statin is selected from amongst atorvastatin, lovastatin, pravastatin, simvastatin, fluvastatin and cerivastatin.

12. A composition according to claim 10, wherein said fibrate is selected from amongst fenofibrate, gemfibrozil, bezafibrate and PPARα agonists.

13. A composition according to claim 1, wherein balaglitazone and said other anti-diabetic compound are presented in two or more separate containers intended for sequentially or concomitantly use.

14. A composition according to claim 1, wherein balaglitazone and said other anti-diabetic compound is presented in a single container.

15. A method of treating conditions benefiting from a decrease in insulin resistance, a reduction of plasma glucose levels, a reduction of plasma fatty acid levels, a reduction of plasma triglyceride levels or a reduction of VLDL levels, the method comprising the administration of balaglitazone in combination with one or more antidiabetic compounds to a patient in need thereof.

16. The method according to claim 15, wherein said condition is selected from amongst type 2 diabetes, dyslipidemia, hyperglycemia, hyperinsulinemia, insulin resistance, obesity, cardiovascular complications, atherosclerosis, hypertension, impaired glucose tolerance, impaired fasting glucose level, increased plasma levels of free fatty acids, increased plasma levels of triglycerides, increased plasma levels of very low density lipoproteins (VLDL).

17. A method for increasing the plasma level of high density lipoproteins at the expense of the plasma level of VLDL, for decreasing the plasma glucose level, for decreasing the plasma level of free fatty acids, for decreasing the plasma triglyceride level, for delaying the progression of impaired glucose tolerance to non-insulin requiring type 2 diabetes, or for delaying the progression of non-insulin requiring type 2 diabetes to insulin requiring type 2 diabetes, the method comprising administering to a patient in need thereof an effective amount of balaglitazone in combination with one or more other anti-diabetic compounds.

18. The method according to claim 15, wherein said other anti-diabetic agent is selected from amongst insulin together with derivative and analogues thereof, insulin secretagogues, insulin sensitizers, biguanides, α-glucosidase inhibitors, potassium channel openers, glucagon antagonists, protein tyrosine phosphatase inhibitors, glucokinase activators, RXR agonists, hormone sensitive lipase inhibitors, glycogen synthase kinase-3 inhibitors, glycogen phosphorylase inhibitors, glucose uptake modulators and lipid lowering compounds.

19. The method according to claim 18, wherein said insulin, insulin analogue or insulin derivative is selected from amongst human insulin, human insulin wherein position B28 is Asp, Lys, Leu, Val or Ala and position B29 is Lys or Pro, B28Lys-B29Pro human insulin, des(B28-B30) human insulin, des(B27) human insulin, des(B30) human insulin, B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28}Pro^{B29} human insulin, B28-N^{ε}-palmitoyl Lys^{B28}Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

20. The method according to claim 18, wherein said insulin secretagogue is selected from amongst sulfonylureas, meglitinides and dipeptidyl peptidase inhibitors.

21. The method according to claim 20, wherein said sulfonylurea is selected from amongst tolbutamide, glibenclamide, gliclazide, glimepiride, glipizid, chlorpropamide, tolazamide and glyburide.

22. The method according to claim 20, wherein said meglitinides is selected from amongst nateglinide and repaglinide.

23. The method according to claim 18, wherein said insulin sensitizer is selected from amongst troglitazone, ciglitazone, pioglitazone, rosiglitazone, isaglitazone, darglitazone, englitazone.

24. The method according to claim 18, wherein said biguanide is metformin.

25. The method according to claim 18, wherein said α-glucosidase inhibitor is selected from amongst voglobose, emiglitate, miglitol and acarbose.

26. The method according to claim 18, wherein said lipid lowering compound is a statin, fibrate or a PPARδ agonist.

27. The method according to claim 26, wherein said statin is selected from amongst atorvastatin, lovastatin, pravastatin, simvastatin, fluvastatin and cerivastatin.

28. The method according to claim 26, wherein said fibrate is selected from amongst fenofibrate, gemfibrozil, bezafibrate and PPARα agonists.

29. The method according to claim 15, wherein the patient is obese.

30. The use of balaglitazone and one or more other anti-diabetic compound in the manufacture of a medicament for the treatment of type 2 diabetes, dyslipidemia, hyperglycemia, hyperinsulinemia, insulin resistance, obesity, cardiovascular complications, atherosclerosis, hypertension, impaired glucose tolerance, impaired fasting glucose level, increased plasma levels of free fatty acids, increased levels of plasma triglycerides, increased plasma levels of very low density lipoproteins (VLDL), or for the increase of the plasma level of high density lipoproteins at the expense of the plasma level of VLDL, for the decrease of the plasma glucose level, for the decrease of the plasma level of free fatty acids, for the decrease in the plasma level of triglycerides, for delaying the progression of impaired glucose tolerance to non-insulin requiring type 2 diabetes, or for delaying the progression of non-insulin requiring type 2 diabetes to insulin requiring type 2 diabetes, optionally in obese patients.

31. The use according to claim 30, wherein the medicament is intended for obese patients.

32. The use according to claim 30, wherein said anti-diabetic compound is selected from amongst insulin together with derivative and analogues thereof, insulin secretagogues, insulin sensitizers, biguanides, α-glucosidase inhibitors, potassium channel openers, glucagon antagonists, protein tyrosine phosphatase inhibitors, glucokinase activators, RXR agonists, hormone sensitive lipase inhibitors, glycogen synthase kinase-3 inhibitors, glycogen phosphorylase inhibitors, glucose uptake modulators and lipid lowering compounds.

33. The use according to claim 32, wherein said insulin, insulin analogue or insulin derivative is selected from amongst human insulin, human insulin wherein position B28 is Asp, Leu, Lys, Val or Ala and position B29 is Lys or Pro, B28Lys-B29Pro human insulin, des(B28-B30) human insulin, des(B27) human insulin, des(B30) human insulin, B29-N^{ε}-myristoyl-des(B30) human insulin, B29-N^{ε}-palmitoyl-des(B30) human insulin, B29-N^{ε}-myristoyl human insulin, B29-NE-palmitoyl human insulin, B28-N′-myristoyl Lys^{B28}Pro^{B29} human insulin, B28-N^{ε}-palmitoyl Lys^{B28}Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

34. The use according to claim 32, wherein said insulin secretagogue is selected from amongst sulfonylureas, meglitinides and dipeptidyl peptidase inhibitors.

35. The use according to claim 34, wherein said sulfonylurea is selected from amongst tolbutamide, glibenclamide, gliclazide, glimepiride, glipizid, chlorpropamide, tolazamide and glyburide.

36. The use according to claim 34, wherein said meglitinides is selected from amongst nateglinide and repaglinide.

37. The use according to claim 32, wherein said insulin sensitizer is selected from amongst troglitazone, ciglitazone, pioglitazone, rosiglitazone, isaglitazone, darglitazone, englitazone.

38. The use according to claim 32, wherein said biguanide is metformin.

39. The use according to claim 32, wherein said α-glucosidase inhibitor is selected from amongst voglobose, emiglitate, miglitol and acarbose.

40. The use according to claim 32, wherein said lipid lowering compound is a statin, fibrate or a PPARδ agonist.

41. The use according to claim 40, wherein said statin is selected from amongst atorvastatin, lovastatin, pravastatin, simvastatin, fluvastatin and cerivastatin.

42. The use according to claim 40, wherein said fibrate is selected from amongst fenofibrate, gemfibrozil, bezafibrate and PPARα agonists.

43. A composition according to claim 2, which is presented in two or more separate containers intended for sequentially or concomitantly use.

44. A composition according to claim 2, which is presented in a single container.

43. The method according to claim 17, wherein said other anti-diabetic agent is selected from amongst insulin together with derivative and analogues thereof, insulin secretagogues, insulin sensitizers, biguanides, α-glucosidase inhibitors, potassium channel openers, glucagon antagonists, protein tyrosine phosphatase inhibitors, glucokinase activators, RXR agonists, hormone sensitive lipase inhibitors, glycogen synthase kinase-3 inhibitors, glycogen phosphorylase inhibitors, glucose uptake modulators and lipid lowering compounds.

44. The method according to claim 17, wherein the patient is obese.
